(19) United States
(12) Patent Application Publication
(10) Pub. No.: US 2006/0173383 A1
(43) Pub. Date: Aug. 3, 2006

(54) Shear-stress microsensor and surgical instrument end tool

(76) Inventors: Daniel Esteve, Ramonville (FR); Frederich Van Meer, Toulouse (FR); Alain Giraud, Toulouse (FR); Nadege Villeroy, Pibrac (FR)

Correspondence Address: YOUNG & THOMPSON, 745 SOUTH 23RD STREET, 2ND FLOOR, ARLINGTON, VA, 22202, US

(21) Appl. No.: 10/532,307

(22) PCT Filed: Oct. 21, 2003
(86) PCT No.: PCT/FR03/03119

§ 371 Date: Aug. 19, 2005

(30) Foreign Application Priority Data
Oct. 22, 2002 (FR) 02/13168

(51) Int. Cl. A61B 5/103

(52) U.S. Cl. 600/587

(57) ABSTRACT

The invention relates to an end tool for a surgical instrument, comprising a tool-holder support (1) made from a rigid material including a flat face (2) or base layer adapted to support a tool, and a surgical tool (11) formed by a stack of elementary layers adapted to be firmly joined to one another so as to form a functional tool unit that can be positioned on and firmly joined to the base layer (2) of the tool-holder support. The surgical tool includes at least one electronic layer (20) which is made using electronics and microelectronics technology and incorporates integrated connections to an electronic and/or light and/or fluid power source, and at least one electronic component (21, 22, 23) for measuring and/or actuating and/or supplying power, and an upper functional layer (33) having a form which is designed to ensure the operation of the tool.

The invention relates to a shear-stress microsensor. It extends to a surgical instrument end tool including such a stress microsensor.

“Microsensor” refers to a sensor produced primarily by micromachining (deposition, etching, cutting, etc.) according to general microelectronic manufacturing technologies, in particular using silicon wafers. A microsensor may therefore have dimensions far larger than one micrometer, corresponding to those of an integrated circuit chip, typically from 1 mm² to 1 cm².

Numerous pressure stress microsensors are already known that permit measurement of stress values applied in directions perpendicular to the principal faces of the microsensors.

Also known are multidirectional stress sensors that permit measurement of stresses in a plurality of directions, in particular three orthogonal spatial directions.

However, in some applications a need is felt for the availability of multidirectional stress microsensors. The technologies and principles used in the known pressure stress microsensors are not compatible with measurement of shear stresses (parallel to the faces of the microsensor).

It is therefore an object of the invention to propose a stress microsensor able to measure shear stresses.

It is also an object of the invention to propose such a microsensor which is simple to manufacture, can be compact (less than 50 mm² and having a thickness of the order of or less than 1 mm) and can measure stresses which may be up to several newtons, in particular up to 3 N.

More particularly, it is an object of the invention to propose a microsensor to be used in a surgical instrument end tool for measuring shear stresses.

A strong tendency in modern surgery is to minimize traumatisms caused to the patient by operative practice. This tendency is well illustrated by laparoscopic practice which consists in intervening only through incisions of small dimensions and working with indirect vision on a video image obtained by a camera and lighting positioned in the working zone. The surgical end tool is then carried by an instrument which passes through the skin via a trocar placed in the incision. The most commonly used method consists, for the surgeon, in working with two instruments (right hand and left hand) while an assistant is responsible for illumination and image capture. The instruments are in the form of 40 cm-long tubes, the tool-carrying part of which enters the body. The exterior part is maneuvered by the surgeon to reach the working zone and to carry out the operations of cutting, cauterizing, stitching, etc. In the most conventional operating method the surgeon manipulates two instruments, basing his actions on images transmitted to him by an endoscopic camera.

However, this recently developed laparoscopic practice is only one step towards the progressive automation of the surgeon's hand movements. For some years numerous research studies have been concerned with remote-controlled surgery in which the surgeon and his assistant interface by means of robots which perform (on their orders) the manipulation of the instrument and the end tool; the surgeon therefore operates remotely with minimum traumatization.

To meet these new requirements end tools have been designed such as the tool with a cutting blade described in the patent application WO 02/07617. This is a tool of conventional type, i.e. a mechanical tool produced by conventional machining or injection technologies, in a passive portion of which a space is formed; an electronic substrate incorporating a plurality of components including measuring sensors is inserted and fixed in the space.

This solution therefore consists in using current passive tools and associating with them measuring and control electronics specifically dedicated to each tool. Although such a solution allows the above-mentioned new requirements to be met in theory, it has proved in practice to lead to prohibitive manufacturing costs for the surgical tools.

In addition, this solution does not permit measurement of shear stresses, an indispensable step in designing end tools for robots. Inventors have found that measurement of pressure stresses is not sufficient in an end tool such as forceps, bistoury, scissors, etc., for correctly incorporating such tools into a reliable, precise and efficient robot.

It is therefore an object of the present invention to mitigate this disadvantage and to provide a multi-purpose surgical end tool compatible with utilization with a surgical robot. More particularly, it is an object of the invention to propose an end tool provided with means for measuring stresses in all useful directions. It is also an object of the invention to propose an “intelligent” end tool able to be produced in large volume with low production costs.

To achieve this, the invention relates to a stress microsensor to be incorporated between two mechanical elements of a kinematic chain and comprising two parallel flat faces, one of which, called the fixed face, is designed to be connected to a first mechanical element such as a support and the other, called the moving face, is designed to be connected to a second mechanical element such as a tool, and comprising, between these two faces, a stress measuring assembly including at least one micromachined layer and adapted to supply an electronic signal representing a stress applied between the moving face and the fixed face, wherein:

the fixed face is firmly joined to a first block of micromachined silicon, called the fixed block,

the moving face is firmly joined to a second block of micromachined silicon, called the moving block,

clearance is provided between the fixed and mobile blocks so as to allow relative translational displacement of these blocks in at least one direction, called the shear displacement direction, parallel to the fixed and mobile faces,

the fixed and mobile blocks are connected to one another by means of a restoring device of micromachined elastically deformable silicon under the effect of relative displacement of the blocks in said shear displacement direction,

it includes an assembly for measuring relative displacements of the blocks in said shear displacement direction that is able to supply a signal representing these displacements and therefore representing the shear stress applied between the moving face and the fixed face.

For some applications use may be made of a microsensor according to the invention adapted to measure shear stresses in only one shear displacement direction, as it may be of interest to reduce the cost and complexity of such a microsensor. This may be the case, in particular, for a forceps designed for monodirectional axial use.

However, there is preferably provided a microsensor according to the invention wherein:

clearance is provided between the fixed and moving blocks so as to allow relative translational displacement of these blocks in all shear displacement directions,

the restoring device is elastically deformable under the effect of relative displacement of the blocks in all shear displacement directions,

the measuring assembly is adapted to measure relative displacements of the blocks in all shear displacement directions and is able to supply a signal representing these displacements and therefore representing the corresponding shear stress applied between the moving face and fixed face.

Furthermore, there is advantageously provided a microsensor according to the invention wherein:

clearance is provided between the fixed and moving blocks so as to allow relative translational displacement of these blocks in at least one direction, called the pressure direction, perpendicular to the fixed and moving faces,

the restoring device is elastically deformable under the effect of relative displacement of the blocks in each pressure direction,

it includes a measuring assembly adapted to measure relative displacement of the blocks in each pressure direction and able to supply a signal representing these displacements and therefore representing the pressure stress applied between the moving face and the fixed face. Thus, a microsensor according to the invention can be tridirectional in translation and can measure stresses in every translational spatial direction.

It should be noted that if a plurality of measuring assemblies are provided for separately measuring the relative pressure displacements of a plurality of distinct zones of the moving block, tipping and tilting stresses can also be measured. Thus, if at least three distinct pressure directions with at least three assemblies for measuring displacements in these three pressure directions are provided, the microsensor according to the invention allows measurement of all tilting movements of the moving block with respect to the fixed block, i.e. a total of five to six axes of movement.

The measuring assembly of a microsensor according to the invention may be realized in various known fashions (electromagnetic, piezoelectric, etc.). Its function is to measure relative displacements and convert them into stress values.

The measuring assembly according to the invention is advantageously of the capacitive type and includes at least one electrode, called the fixed electrode, firmly joined to the fixed block and at least one electrode, called the moving electrode, firmly joined to the moving block, the electrodes being arranged opposite one another so as to form between them a capacitance the value of which varies during relative displacements of the blocks in the shear directions.

According to the invention each electrode of a pair of these opposed (fixed and moving) electrodes is advantageously formed by a comb of strips of conductive material extending parallel to one another and to the fixed and moving faces, and extending orthogonally to a shear direction in which this pair of electrodes allows the relative displacements of the blocks to be measured. According to the invention there is advantageously provided at least one first pair of electrodes adapted to detect relative displacements along a first shear axis (x) and at least one second pair of electrodes adapted to detect relative displacements along a second shear axis (y) perpendicular to the first shear axis (x). The advantage of such a capacitive measuring assembly is to offer no resistance or friction and to be completely stable with respect to temperature.

In an advantageous embodiment there is also provided a microsensor according to the invention wherein the fixed block includes at least one rectangular recess for receiving the rectangular moving block, and wherein it includes four corner elbow levers of micromachined silicon that are elastic in deflection, each elbow lever having one end connected to a side wall of the recess and another end connected to a side wall of the moving block orthogonal to said side wall of the recess, so that this elbow lever is interposed between a corner of the recess and an opposed corner of the moving block and is able to be deformed elastically in deflection when the moving block is displaced in a shear direction with respect to the recess.

The invention also relates to an end a tool of a surgical instrument, comprising:

a tool-holder support made of a rigid material including a flat face, called the base layer, adapted to support a tool,

a surgical tool composed of a stack of elementary layers firmly joined to one another so as to form a functional tool unit fixed to the base layer of the tool-holder support, and including at least one layer forming a stress microsensor and a functional end layer having a form designed to ensure the operation of the tool.

There is provided a tool according to the invention wherein the surgical tool includes at least one stress microsensor according to the invention. The surgical tool may include a single microsensor or a plurality of microsensors mounted in parallel to increase the stress values that can be measured.

According to the invention said surgical tool also advantageously includes at least one micromachined layer, called the electronic layer, incorporating a connector for connection to an electronic and/or light and/or fluid power source, and at least one electronic function for signal processing and/or measurement and/or actuation and/or energy supply.

The fundamental concept of the invention has therefore been to produce an end tool composed, firstly, of a mechanical support designed to be integrated functionally in a robotized or manual surgical instrument and, secondly, of a tool having the conventional functions of a surgical tool (forceps, scissors, bistoury, etc.), a shear stress microsensor and functions for signal processing, measurement, control, etc., which ensure the convenience of the surgeon and the performance of the system, said tool being produced by stacking elementary layers while using, for example, general microelectronic manufacturing technologies (integration on silicon) and the assembly technology called hybrid technology, so as to form a monolithic tool unit.

Such a concept has the fundamental advantage of enabling “intelligent” tools to be manufactured jointly, so that they can be produced in large volume with low production costs.

According to another advantageous characteristic of the invention, the surgical tool includes a support layer designed to be firmly joined to the base layer of the tool-holder support and including a connector to be connected, firstly, to the connector of each electronic layer and, secondly, to an electrical and/or light and/or fluid power source.

Such a support layer allows a connecting “bridge” to be produced that isolates the surgical instrument from stresses acting on the energy connection elements linking said tool to the energy sources. In addition, it forms a base platform facilitating the production of the tool unit.

According to another advantageous characteristic of the invention, the surgical tool includes an interface layer adapted to be firmly attached below the functional layer and incorporating the components for energy transfer between the external medium and the electronic layer.

In addition, the surgical instrument advantageously includes pins extending through superposed openings formed in the different layers of said tool and designed to be fixed into openings formed in the base layer of the tool-holder support.

As an example of an advantageous embodiment, the end tool according to the invention may consist of an end tool consisting of a forceps formed by two assemblies comprising the tool-holder support/surgical instrument according to the invention, the tool-holder supports of which are each provided on a prolongation of their base layer with a lug orthogonal to said base layer for the articulation of the forceps.

In addition, in order to form a forceps consisting of a bistoury, the functional layer of each surgical tool incorporates at least one electrode flush with the upper face of said functional layer, the interface layer including a conductive component for supplying each electrode with power.

Another advantageous embodiment consists of a bistoury having one blade or a scissors blade including a functional layer in the form of a blade having a longitudinal side face with a beveled profile forming a longitudinal cutting edge.

In addition, in order to form a bipolar bistoury the functional layer forms a bipolar blade provided with a portion of thickness made of electrically conductive material, the interface layer including a conductive component for supplying said conductive portion with power.

Other characteristics, objects and advantages of the invention will be apparent from the following detailed description with reference to the appended drawings representing, by way of non-limiting examples, two preferred embodiments, in which drawings:

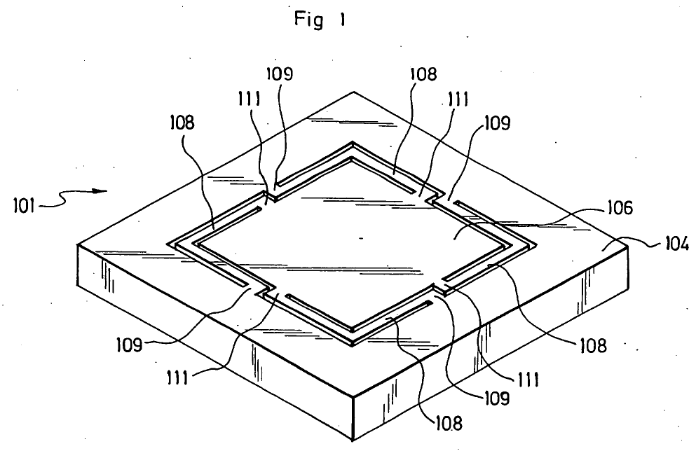

FIG. 1 is a schematic perspective view showing the principle of a test body (mechanical part comprising a fixed block and a moving block) of a microsensor according to the invention,

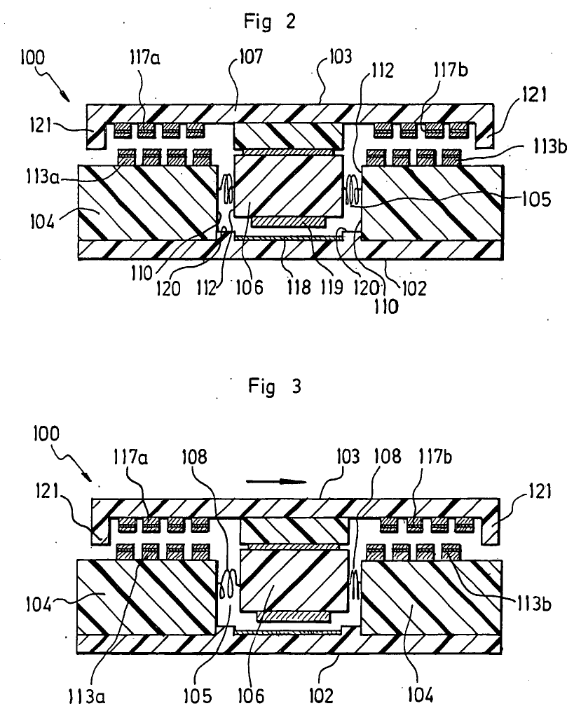

FIGS. 2 and 3 are schematic sectional views showing the principle of a microsensor according to the invention, respectively at rest and after the application of a shear stress,

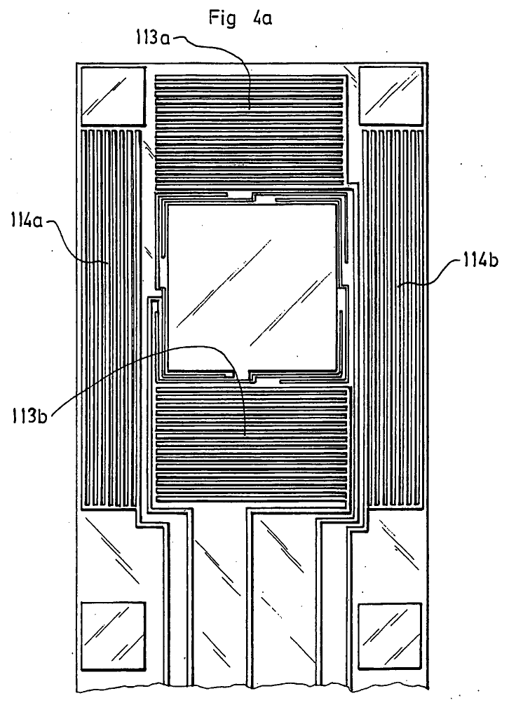

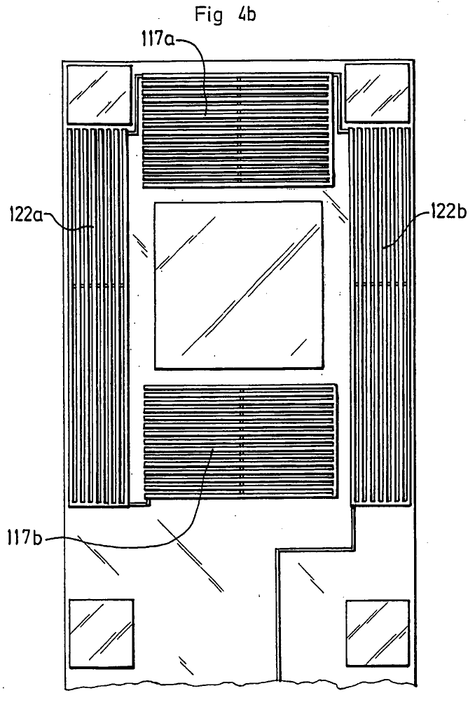

FIG. 4a is a top view of an exemplary embodiment of a lower part including a test body of a microsensor according to the invention, FIG. 4b being a view from below of an exemplary embodiment of the corresponding upper part of the microsensor according to the invention,

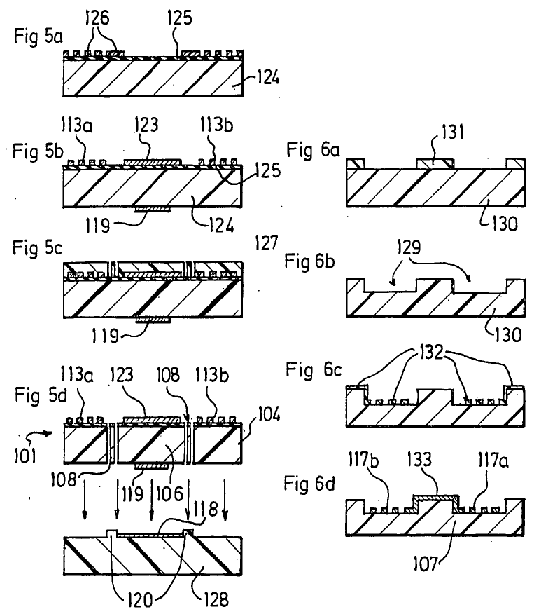

FIGS. 5a to 5d are schematic sectional views illustrating different successive steps of a method for producing the lower part of a microsensor shown in FIG. 4a,

FIGS. 6a to 6d are schematic sectional views illustrating different successive steps of a method for producing the upper part of a microsensor shown in FIG. 4b,

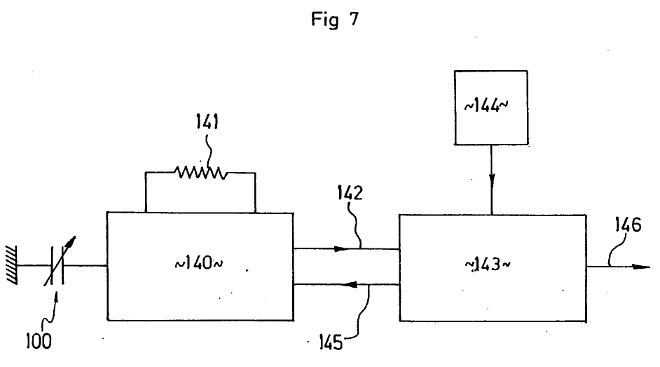

FIG. 7 is a block diagram of an example of an electronic circuit for processing the signal of a microsensor according to the invention,

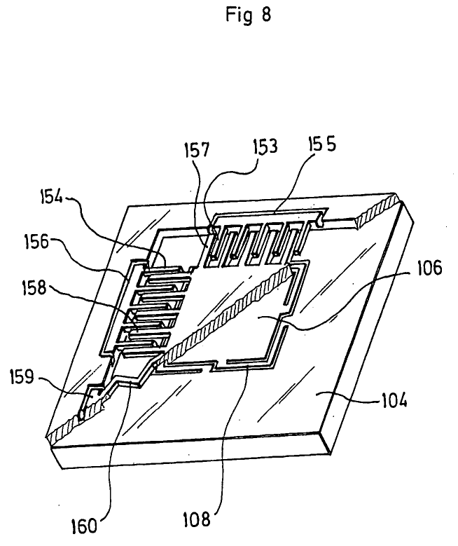

FIG. 8 is a schematic partially cut-away perspective view of a variant of a microsensor according to the invention,

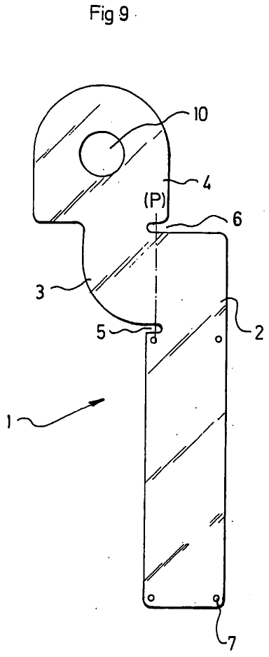

FIG. 9 is a plan view of a tool-holder support according to the invention before folding,

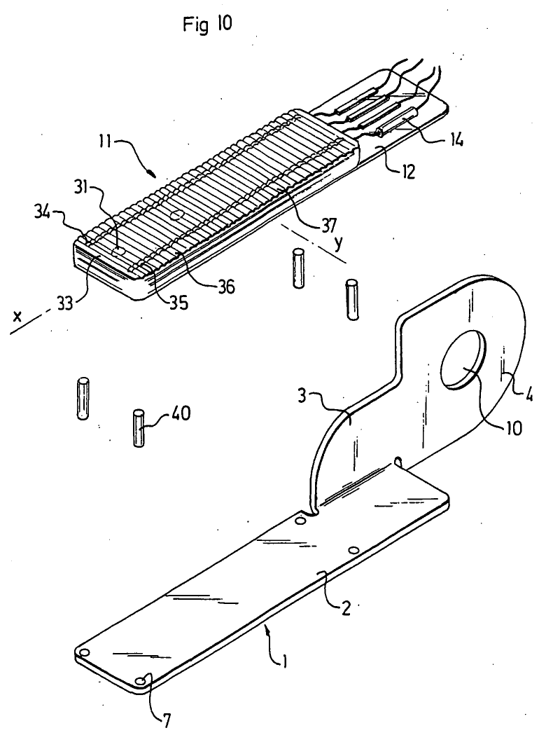

FIG. 10 is an exploded perspective view showing the elements of one of the jaws of an electric bistoury according to the invention,

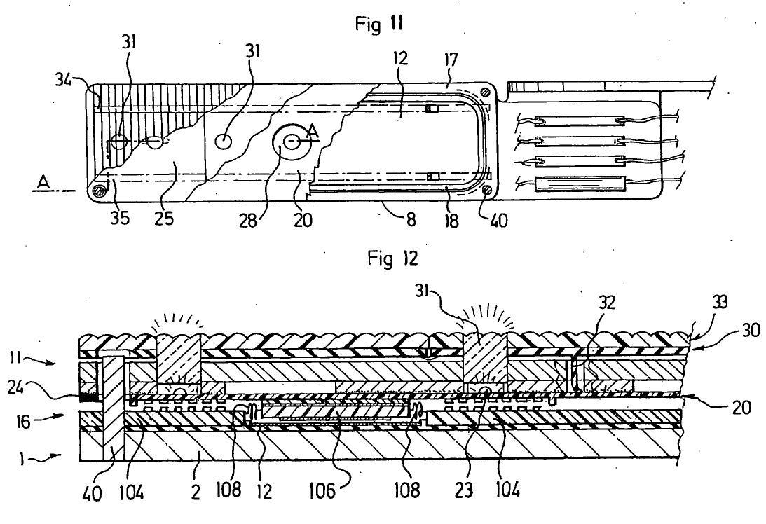

FIG. 11 is a partially cut-away top view of said jaw,

FIG. 12 is a longitudinal section through a broken plane A of said jaw,

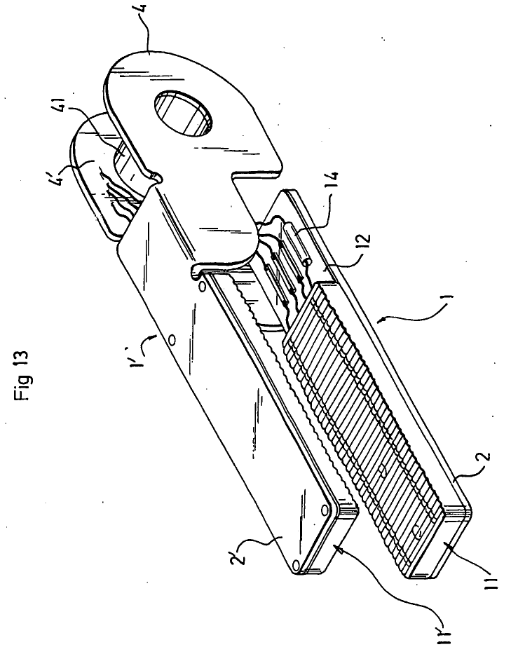

FIG. 13 is a perspective view of an electric forceps according to the invention formed by two jaws as shown in FIGS. 10 to 12,

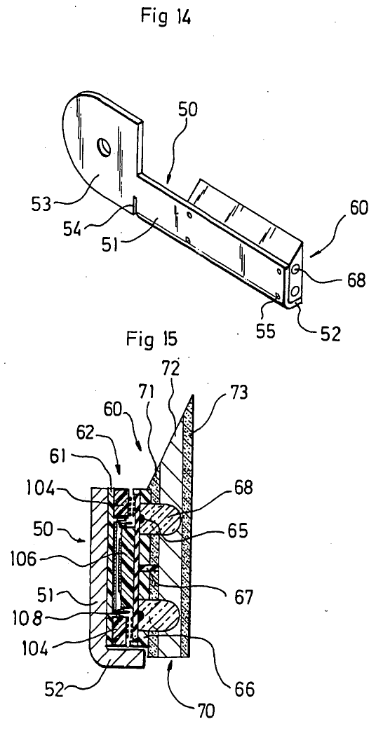

FIG. 14 is a perspective view of a bistoury having a bipolar blade according to the invention, and

FIG. 15 is a cross-section through this bistoury having one blade.

FIGS. 2 and 3 show a microsensor 100 according to the invention, comprising a test body 101 the principle of which is shown in FIG. 1. The microsensor 100 includes two parallel flat faces comprising one fixed face 102 designed to be connected to a first mechanical element such as a support, and a moving face 103 designed to be connected to a second mechanical element such as a tool or to a functional layer forming a tool. The two, fixed and mobile, faces 102, 103 are parallel to one another and flat. The fixed face 102 forms the base of the test body 101. The test body 101 includes a micromachined block of silicon 104, called the fixed block 104, all the elements of which are firmly attached to the face 102. This fixed block 104 takes the general form of a frame and defines a rectangular or square central recess 105 for receiving a moving block 106 having a shape conjugate to that of the recess 105, i.e. rectangular or square, the dimensions of which are smaller and parallel to the planes of the fixed 102 and moving faces 103, so that this moving block 106 can move parallel to the fixed 102 and moving 103 faces inside the recess 105. The moving block 106 is also a block of micromachined silicon firmly attached to an upper wafer 107 forming the moving face 103.

The moving block 106 is connected to the fixed block 104 by four corner elbow levers 108 formed of micromachined silicon and extending between the opposed walls the fixed block 104 and the moving block 106 in the gap separating them. Each elbow lever 108 includes an end 109 integral with the side wall 110 of the recess 105 of the opposed fixed block 104. The other end 111 of the elbow lever 108 is connected to the side wall 112 of the moving block 106 which extends orthogonally to the side wall 110 of the recess 105. In this way the elbow lever 108 is interposed between a corner of the recess 105 of the fixed block 104 and an opposed corner of the moving block 106, and is able to be deformed elastically in deflection when the moving block 106 is displaced with respect to the recess 105 in any shear direction parallel to the fixed 102 and moving 103 faces. Between the two ends 109, 111 the elbow levers 108 are independent both of the moving block 106 and of the recess 105 in the fixed block 104. In addition, lateral clearance is provided on each side of each elbow lever 108, i.e. on one side with respect to the recess 105 and on the other side with respect to the moving block 106, so that deflections of the elbow lever 108 are permitted. The dimension of these lateral clearances determines the amplitude of the shear displacement of the moving block 106 with respect to the fixed block 104. In FIGS. 2, 3, 11, 14 the elbow levers 108 are represented for clarity by springs and are not shown realistically.

Furthermore, around the recess 105 the fixed block 104 has four electrode combs 113a, 113b, 114a, 114b. Each comb is formed by a plurality of electrically conductive strips, for example, of gold, parallel to one another and spaced from one another, which strips are each connected jointly by one end to a connecting track 115a, 115b, 116a, 116b respectively, which connects the electrical current from the different strips of the comb. Two combs 114a, 114b are arranged on either side of the recess 105 parallel to its opposed longitudinal sides for measuring shear forces in a shear direction orthogonal to said combs 114a, 114b. Two other combs 113a, 113b are arranged on either side of the recess parallel to the lateral sides of the recess 105 to measure a stress in a longitudinal shear displacement direction orthogonal to said combs 113a, 113b.

The upper moving wafer 107 associated with the moving block 104 is also provided with four electrode combs 117a, 117b, 122a, 122b similar to those of the fixed block 104 and arranged on the lower face of said wafer 107 so as to be located respectively opposite the four combs 113a, 113b, 114a, 114b of the fixed block 104. In the sectional FIGS. 2 and 3 only the combs 117a, 117b designed to be located above the combs 113a, 113b are shown. The wafer 107 is associated with the moving block 106 in such a way that the combs 117a, 117b, 122a, 122b carried by it are maintained at a distance from the corresponding opposed electrode combs 113a, 113b, 114a, 114b of the fixed block 104, so that a capacitive effect is produced between the different opposed electrode combs. The combs 117a, 117b, 122a, 122b are themselves likewise connected to electrical connecting tracks (not shown), the different strips of each comb being connected to the same connecting track.

As can be seen in FIGS. 2 and 3 (which are only schematic representations), when the moving block 106 is displaced in a shear direction parallel to the faces 102, 103 under the effect of a shear stress F, the value of the capacitance existing between the different electrode combs varies, since the opposed surface area of the conductive electrodes is no longer the same. This variation of capacitance provides a precise measure of the displacement value induced by the stress F. Because the corner elbow levers 108 are elastic restoring elements and have a predetermined stiffness, the displacement value also furnishes a value of the stress F.

In addition, the bottom of the recess 105 is covered with a conductive metal layer 118, for example of gold, and the lower face of the moving block 106 is also covered with a conductive metal layer 119, for example of gold, so that a capacitive effect is also produced between the layer on the bottom 118 and the layer on the lower face 119. The two opposed metal layers 118, 119 which form a capacitance are also themselves connected to conductive connecting tracks.

The capacitance formed in this way may be used to measure pressure stresses in a direction orthogonal to the fixed 102 and moving 103 faces. The bottom layer 118 extends over an area greater than that of the layer 119 on the moving block 106, so that the value of the capacitance formed between them does not change during shear displacements of the moving block 106. However, this capacitance is modified if the moving block 106 moves closer to the bottom of the fixed block 104 under the effect of a pressure stress applied orthogonally to the moving face 103. As this happens, the elbow levers 108 have an elastic restoring effect on the moving block 106 in the pressure direction. They are also elastic in deflection in this direction normal to the fixed 102 and mobile 103 faces.

To achieve this, the clearance between the different electrode combs and between the bottom 118 of the recess 105 and the lower face 119 of the moving block 106 must be sufficient to allow sufficient amplitude of displacement orthogonal to the faces 102, 103. If that is the case, when a pressure stress is applied between the faces 102, 103, this pressure stress induces displacement of the moving block 106 with respect to the fixed block 104 and therefore a modification of the distance between the electrode combs on the one hand and the conductive layers 118, 119 of the bottom of the fixed block 104 and of the lower face of the moving block 106, on the other.

In a variant (not shown), the bottom layer 118 fixed to the fixed block 104 may be divided into at least three distinct parts insulated from one another—for example, into four squares or rectangles each forming one of the corners of said layer 118. Each of the parts is connected to its own connecting track, so that four different, independent capacitances independently measuring the stresses in the four corners of the layers 118, 119 are formed. In this way stresses in four different pressure directions can be measured and a measure can be obtained of the tilt stresses on the mobile block 106 with respect to the fixed block 104.

Lateral stops 120 of insulating silicon are provided at the periphery of the conductive layer 118 at the bottom of the fixed block 104, extending above its free face to a height greater than the thickness of the layer 118, so as to limit the displacement travel of the moving block 106 towards the bottom of the fixed block 104 and to prevent contact through compression towards one another of the conductive layers 118, 119 and between the opposed electrode combs.

Also provided are lateral stops 121 integral with the upper moving wafer 107 and extending downwardly from the lower face of said wafer 107 to a height greater than that of the conductive strips forming the electrode combs 117a, 117b, 122a, 122b, so as also to prevent contact through compression towards one another of the opposed electrode combs.

As can be seen in FIG. 4b, the different strips of the combs 117a, 117b, 122a, 122b, as well as the different combs, are jointly connected by conductive wires or conductive tracks, together with the conductive layer 119 of the lower face of the moving block 106, to the same connecting track which can be connected to ground. Thus, all the electrodes carried by the moving block of the microsensor are connected to ground.

It should be noted that the terms the “fixed” and “moving” used with reference to the faces 102, 103 and the blocks 104, 106 mean that the two elements are movable with respect to one another, without what is called the “fixed” element necessarily being actually fixed within a terrestrial frame of reference. Thus, there is no reason why the microsensor should not be used in a kinematic chain in which the moving block and the moving face remain actually fixed in relation to a terrestrial reference frame, while the fixed block 104 and the fixed face 102 are subjected to movements with respect to this terrestrial reference frame.

FIGS. 5a to 5d illustrate different successive steps in manufacturing an exemplary embodiment of the fixed block 104 of a microsensor according to the invention.

The first step, shown in FIG. 5a, starts with a wafer of silicon 124 covered with a layer of silicon oxide 125, then a mask of photolithographic resin 126 having the format of the electrodes to be formed on the fixed block 104 (combs 113a, 113b, 114a, 114b), and an upper layer 123 for linking the moving block 106 to the upper moving wafer 107.

With the aid of said mask a layer of titanium is deposited first, then a layer of gold (the titanium serving to anchor the gold), then the photolithographic resin is eliminated to obtain the result shown in FIG. 5b.

The assembly is covered with a new layer 127 of photolithographic resin having the format of the voids to be formed to produce the elbow levers 108, as shown in FIG. 5c. After performing deep RIE etching to form the clearances between the elbow levers 108 and the moving block 106 and the fixed block 104, the result shown in FIG. 5d is obtained, including the test body 101 provided with electrodes.

As is shown in FIG. 5b, a conductive layer of gold forming the lower conductive layer 119 of the moving block 106 has also been deposited on the opposed face of the silicon wafer 124 by means of an appropriate photolithographic mask.

During the step shown in FIG. 5d, said test body is bonded to a lower base layer 128 provided in advance with lateral stops 120 and with the conductive layer 118 forming the bottom of the recess 105.

FIGS. 6a to 6d show different successive steps in producing the upper moving wafer 107 and the electrodes carried thereby. Starting from a silicon wafer 130, a layer of photolithographic resin 131 is deposited that forms a mask having the format of the recesses 129 to be formed in the thickness of said plate 130 to receive the electrode combs 117a, 117b, 122a, 122b. After RIE etching the result shown in FIG. 6b is obtained. A new layer of resin 132 is deposited, as shown in FIG. 6c, having the format of the electrodes to be produced to form the electrode combs and a conductive layer 133 for connection to the moving block 106. After the deposition of a layer of titanium and gold the result shown in FIG. 6d is obtained, formed by the moving wafer 107 provided with the electrode combs 117a, 117b, 122a, 122b.

It is then sufficient to assemble this wafer 107 on the mounting obtained as shown in FIG. 5d by soldering together the conductive layers 133 of the central terminal of the wafer 107 and the upper conductive layer 123 of the moving block 106, so that this moving block 106 is permanently associated with the moving wafer 107.

In the variant shown in FIG. 8, the microsensor according to the invention is produced from a single initial wafer formed by an SOI (silicon, silicon oxide and conductive doped silicon) substrate. Deep RIE etching is first performed in the rear face of the silicon layer as far as the layer of silicon oxide, the etching having the format of the elbow levers 108 separating the moving block 106 from the fixed block 104. Etching is then performed on the front face in the conductive silicon layer in the format of the electrode combs to be produced. The intermediate layer of oxide SiO₂ is then etched using anisotropic etching so as to free the electrode combs 153, 154, 157, 158 from the oxide layer. In this variant the electrode combs 153, 154 joined to the fixed block 104 via the oxide layer which carries them are insulated electrically from this fixed block 104 by means of a peripheral groove 155, 156 formed in the conductive layer at the same time as the combs 153, 154. In addition, the electrode combs 157, 158 joined to the moving block 106 have electrodes disposed adjacent (in the lateral direction) to those of the combs 153, 154 of the fixed block 104, but intermeshed with these electrodes. In this way, the electrodes are not superposed, as in the variant shown in FIGS. 2 and 3, but juxtaposed.

The electrode combs 157, 158 joined to the moving block 106 are all jointly connected to a connecting terminal 159 etched in the fixed block 104 and insulated electrically from the latter by means of a flexible strip 160 in the form of a jagged line permitting relative displacements of the moving block 106 with respect to the terminal 159. In FIG. 8, the layers of SiO₂ and conductive doped Si are partially cut away along a diagonal. The electrode combs 153, 154 of the fixed block 104 and those 157, 158 of the moving block 106 are formed using the same mask.

In this way, in this variant both the test body 101 and the electrode combs have been produced starting from a single substrate.

The assembly can then be applied to a base layer such as that 128 shown in FIG. 5d to form the capacitance for measuring pressure stresses.

FIG. 4a shows a top view of the lower part carrying the fixed block 104 of the microsensor obtained in the step shown in FIG. 5d. FIG. 4b shows a top view of the upper part comprising the moving wafer 107 of the microsensor as obtained in the step shown in FIG. 6d.

FIG. 7 is an electrical block diagram showing the principle for processing a signal emitted from the microsensor according to the invention. The microsensor 100 may be symbolized by a variable capacitance 100, one of the plates of which is connected to ground (electrode joined to the mobile wafer 107) while the other is connected to the input of a monostable circuit 140 which allows the variable capacitance 100 to be charged via a resistor 141. According to the value of the capacitance of the microsensor 100, the RC circuit thus formed takes a longer or shorter time to be charged. When the capacitance is charged, the monostable circuit 140 sends an end-of-charge signal 142 to a microcontroller 143. This high-speed microcontroller 143 is synchronized by a clock 144 and sends a signal 145 to the monostable circuit 140 to release the charge of the variable capacitance 100. In this way the microcontroller 143 can calculate the total charging duration and convert it into a capacitance value supplied at a digital output 146. On the basis of each capacitance value supplied by the different electrodes of the microsensor, a logic circuit external to the microsensor can calculate the corresponding stress values.

To do this, after the microsensor has been mounted on the tool, a calibration phase enables a standardization matrix representing the mapping of stress/capacitance value in each case (in two or three dimensions depending on whether or not pressure stresses are measured) to be recorded in a read-only memory. Starting from a vector of capacitance values, this matrix allows the corresponding vector of stresses to be obtained.

As can be seen, such a microsensor enables stress measurements to be supplied for all shear directions, but also for pressure, i.e. in practice for all spatial directions, even for tilt, at least within a predetermined range of amplitudes corresponding to the clearance that can exist between the electrodes forming the measuring capacitances.

In this way, the inventors have noted with surprise that micromachined silicon could indeed enable the very efficient production of such a test body which is able to measure stress of relatively high value, that may be of several newtons, in particular up to 3 N (300 g-force). Such a microsensor, of extremely small dimensions, can be integrated as an elementary layer in a tool such as a surgical tool formed by a plurality of layers produced according to microelectronic technologies. In this way the extremely compact microsensor is compatible with the production of a tool which is itself of very small dimensions, for example, of the order of 8 mm long, 3.5 mm wide and 1 mm thick overall.

In FIGS. 1 to 6d the thickness and length dimensions are not shown to scale for purposes of illustration (thicknesses are increased and lengths reduced in relation to reality).

The different connecting tracks connected to the different electrodes of the microsensor are connected electrically to an electronic circuit that can be produced by integration on silicon either beside the microsensor 100, i.e. with at least one silicon substrate in common, or in an upper or lower layer.

In a variant (not shown) the fixed face 102 of the microsensor may also carry contacts or connecting pins to allow the microsensor to be mounted in a simple manner on a support in the manner of an integrated circuit.

Such a microsensor may, in particular, be used for producing an end tool of a surgical instrument as described below.

The two end tools for surgical instruments shown in FIGS. 13 and 14 comprise “intelligent” tools designed to be manufactured jointly. These end tools are each composed of a tool-holder support, produced in the example shown by folding previously machined metal sheets, and of a tool unit produced by stacking elementary layers using assembly and processing technologies known as hybrid technologies.

To begin with, FIG. 9 shows the tool-holder support 1 of one of the jaws of an electric forceps as shown in FIG. 13, or an electric bistoury as shown in FIG. 14.

This tool-holder support 1 is formed by a micromachined sheet-metal part including a first rectangular portion 2, an intermediate lateral portion 3 in the form of a quadrant so formed that one of its bases extends in a co-linear manner in the lateral prolongation of one of the short sides of the rectangular portion 2, and a third portion 4 of semi-oval shape extending in the prolongation of the above-mentioned base of the intermediate portion 3. In addition, two transverse notches 5, 6 are formed respectively at the junction between the rounded edge of the intermediate portion 3 and the corresponding longitudinal edge of the rectangular portion 2, and at the junction of the second and third portions 3, 4, so as to define a folding axis (P) enabling said second and third portions to be folded down so that they extend in a plane perpendicular to the faces of the first portion 2, as shown in FIG. 9.

In this way the rectangular portion 2 forms after folding a support face for the tool unit described hereinafter, extending between the notch 5 and the opposed side edge of said rectangular portion.

Four openings 7 formed at each of the four corners of the support are first drilled therein.

A circular central opening 10 is formed in the third portion 3 for the articulation and actuation of the tool by external motorization or manual systems.

The tool unit 11 according to the invention includes a support layer 12 of a biocompatible material or a two-component material designed to form a biocompatible contour, which has dimensions conjugate with those of the rectangular portion 2 of the tool-holder support 1.

This support layer 12, that is adapted to be joined firmly to the rectangular portion 2 of the tool-holder support 1, includes openings opposite each of the openings 7 in said rectangular portion.

In addition, the connector 14 for connecting said tool unit 11 is attached to the support layer 12, which is designed to form a platform during manufacture of the tool unit.

The second layer 16 of the tool unit 11 includes a microsensor 100 according to the invention for measuring shear stresses and designed to permit measurement of stresses exerted on said tool unit in the plane of the two orthogonal shear axes (x, y) which are parallel to the axes of symmetry of the support face of the tool-holder support 1.

The fixed face 102 of the microsensor 100 is fixed rigidly to the support layer 12, for example by bonding. The moving face 103 of the microsensor 100 is fixed rigidly to a third layer 20, itself joined firmly to the last layer 33 of the tool unit 11 which ensures the operation of the tool, in the present example a jaw of a forceps.

The third layer 20 of the tool unit 11 consists of an electronic layer made using electronic and microelectronic technologies and performing other functions of measurement and control, which for this purpose incorporates microsensors for measuring temperature, displacement, biochemical characteristics, etc., together with microactuators driven, in particular, mechanically or hydraulically, and proximity electronics for signal processing and control.

The electronic layer 20 incorporates, for example, light sources such as 23 mounted on said electronic layer and comprising, for example, either simply light emitting diodes for the single purpose of illumination or light emitting/receiving diodes for purposes, in particular, of proximity detection, tissue characterization and/or tissue presence detection.

This electronic layer 20 also incorporates a sensor 24 for measuring biochemical characteristics which is mounted level with the front edge of the electronic layer 20.

The fourth layer 30 consists of an interface or energy transfer layer made of a biocompatible material and incorporating components for energy transfer between the electronic layer 20 and the external medium.

In the present example, this interface layer 30 includes light wells 31 of transparent material arranged so as to be positioned in each case perpendicularly to a light source 23. This interface layer 30 also incorporates conductive links 32 for electrical connection to the electronic layer 20.

The fifth and last layer 33 of the tool unit 11 consists of the functional layer ensuring the operation of the tool and made of a plastics or metallic material.

The functional layer 33 includes a rippled upper surface. In the example illustrated the functional layer 33 is a single part and is associated with a single microsensor 100 according to the invention.

In a variant (not shown) the functional layer 33 might be divided longitudinally into a plurality of sections able to move freely with respect to one another. In order to permit this free movement of each of the sections of the upper layer 33, the interface layer 30 is also divided longitudinally into a plurality of sections. Each section is associated solidly with the moving face of a microsensor, the tool unit 11 including as many microsensors according to the invention as there are independent sections. Different stresses may therefore be measured on different parts of the jaw formed by this tool unit 11.

The functional layer 33 also includes two longitudinal slots within each of which is mounted an electrode 34, 35 flush with the upper face of said functional layer, supplied electrically via one of the connecting links 32 of the interface layer 30 and designed to form a sliding contact able to absorb the vertical displacements of this functional layer 33.

Finally, the functional layer includes openings each able to house a light well 31 so formed as to be flush with the upper face of said functional layer.

To facilitate the assembly of the various above-described layers forming the tool unit 11, and the fixing of said tool unit to the tool-holder support 1, these layers include opposed openings arranged to form bores aligned with the openings 7 of said tool-holder support, each of which bores is able to receive an assembly pin 40 which, however, is so adapted as not to prevent the shear and compression movements necessary for stress measurement.

FIG. 13 shows an electric forceps composed of two jaws 1-11, 1′-11′ as described above and arranged in inverse positions, the lugs 4, 4′ of the tool-holder supports 1, 1′ being connected by a hinge axis 41 permitting relative pivoting movement of said jaws actuated by external motorization or a manual system.

The second tool shown in FIGS. 14 and 15 consists of a bistoury having one blade or a scissors blade.

Like the preceding tool, it includes, firstly, a tool-holder support 50 formed by a sheet-metal part having, with regard to this tool, a first rectangular portion 51 edged longitudinally by a longitudinal flange 52 perpendicular to the rectangular portion 51 and prolonged by a semi-oval lug 53.

As previously, the rectangular portion 51 includes a notch 54 to permit the folding of the flange 52, and openings 55 for the assembly of pins 40.

The tool unit 60 includes a first support layer 61 and a second layer 62 for measuring shear stresses like those described above.

The tool unit 60 also includes two lighting diodes such as 65.

The tool unit 60 also includes an interface layer 66 including links 67 for electrical conduction and two light guides 68 of semi-oval section, each extending opposite a diode 65 and disposed longitudinally on said interface layer.

The tool unit 60 includes, finally, a functional layer 70 forming a bipolar blade and consisting of three superposed layers comprising a conductive layer 72 supplied with current by links 67 which is sandwiched between two layers 71, 73 made of a non-conductive material.

In addition, two longitudinal grooves are formed in the lower face of the functional layer 70 and are shaped to house the light guides 68 so as to supply light beams at the end face of the tool.

Finally, to form the cutting edge of the blade, the functional layer has a longitudinal side face having a beveled profile.

1. A stress microsensor designed to be incorporated between two mechanical elements of a kinematic chain and comprising two parallel flat faces, one of which, called the fixed face (102), is designed to be connected to a first mechanical element such as a support and the other, called the moving face (103), is designed to be connected to a second mechanical element such as a tool, and comprising, between these two faces (102, 103), a stress-measuring assembly including at least one micromachined layer and adapted to supply an electronic signal representing a stress applied between the moving face (103) and the fixed face (102), wherein:
the fixed face (102) is firmly joined to a first block of micromachined silicon, called the fixed block (104),
the moving face (103) is firmly joined to a second block of micromachined silicon, called the moving block (106),
clearance is provided between the fixed (104) and mobile (106) blocks so as to allow relative translational displacement of these blocks (104, 106) in at least one direction, called the shear displacement direction, parallel to the fixed (102) and moving (103) faces,
the fixed (104) and moving (106) blocks are connected to one another by means of a restoring device (108) of micromachined elastically deformable silicon under the effect of relative displacement of the blocks (104, 106) in said shear displacement direction,
it includes an assembly (113a, 113b, 114a, 114b, 117a, 117b, 122a, 122b) for measuring relative displacements of the blocks in said shear displacement direction that is able to supply a signal representing these displacements and therefore representing the shear stress applied between the moving face (103) and the fixed face (102).

2. The microsensor as claimed in claim 1, wherein:
clearance is provided between the fixed (104) and moving (106) blocks so as to allow relative translational displacement of these blocks in all shear displacement directions,
the restoring device (108) is elastically deformable under the effect of relative displacement of the blocks in all shear displacement directions,
the measuring assembly (113a, 113b, 114a, 114b, 117a, 117b, 122a, 122b) is adapted to measure relative displacements of the blocks (104, 106) in all shear displacement directions and is able to supply a signal representing these displacements and therefore representing the corresponding shear stress applied between the moving face (103) and fixed face (102).

3. The microsensor as claimed in claim 1, wherein:
clearance is provided between the fixed (104) and moving (106) blocks so as to allow relative translational displacement of these blocks (104, 106) in at least one direction, called the pressure direction, normal to the fixed (102) and moving (103) faces,
the restoring device (108) is elastically deformable under the effect of relative displacement of the blocks (104, 106) in each pressure direction,
it includes a measuring assembly (118, 119) adapted to measuring relative displacements of the blocks in each pressure direction and able to supply a signal representing these displacements and therefore representing the pressure stress applied between the moving face (103) and the fixed face (102).

4. The microsensor as claimed claim 1, wherein the measuring assembly (113a, 113b, 114a, 114b, 117a, 117b, 122a, 122b) is of the capacitive type and includes at least one electrode, called the fixed electrode (113a, 113b, 114a, 114b), firmly joined to the fixed block (104) and at least one electrode, called the moving electrode (117a, 117b, 122a, 122b), firmly joined to the moving block (106), the electrodes being arranged opposite one another so as to form between them a capacitance the value of which varies during relative displacements of the blocks (104, 106) in the shear directions.

5. The microsensor as claimed in claim 4, wherein each electrode of a pair of opposed fixed (113a, 113b, 114a, 114b) and moving (117a, 117b, 122a, 122b) electrodes is formed by a comb of strips of conductive material extending parallel to one another and to the fixed (102) and moving (103) faces, and extending orthogonally to a shear direction in which this pair of electrodes allows the relative displacements of the blocks (104, 106) to be measured.

6. The microsensor as claimed in claim 5, wherein it includes at least one first pair of electrodes (113a, 113b, 117a, 117b) adapted to detect relative displacements along a first shear axis (x) and at least one second pair of electrodes (114a, 114b, 122a, 122b) adapted to detect relative displacements along a second shear axis (y) perpendicular to the first shear axis (x).

7. The microsensor as claimed in claim 1, wherein the fixed block (104) includes at least one rectangular recess (105) for receiving the rectangular moving block (106), and wherein it includes four corner elbow levers (108) of micromachined silicon that are elastic in deflection, each elbow lever (108) having one end connected to a side wall of the recess (105) and another end connected to a side wall of the moving block (106) orthogonal to said side wall of the recess (105), so that this elbow lever (108) is interposed between a corner of the recess (105) and an opposed corner of the moving block (106) and is able to be deformed elastically in deflection when the moving block (106) is displaced in a shear direction with respect to the recess (105).

8. An end tool of a surgical instrument comprising:
at least one tool-holder support (1, 50) made of a rigid material including a flat face, called the base layer (2, 51), adapted to support a tool,
at least one surgical tool (11, 60) composed of a stack of elementary layers firmly joined to one another so as to form a functional tool unit fixed to the base layer (2, 51) of the tool-holder support, and including at least one layer forming a stress microsensor and a functional end layer (33, 70) having a form designed to ensure the operation of the tool, wherein the surgical tool includes at least one stress microsensor (100) as claimed in claim 1.

9. The end tool as claimed in claim 8, wherein said surgical tool also includes at least one micromachined layer, called the electronic layer (20), incorporating a connector for connection to an electronic and/or light and/or fluid power source, and at least one electronic function for signal processing and/or measurement and/or actuation and/or power supply.

10. The end tool as claimed in claim 8, wherein the surgical tool (11; 60) includes a support layer (12; 61) designed to be fixed to the base layer (2; 51) of the tool-holder support (1; 50) and including a connector (14) for connection, firstly, to the connector of each electronic layer and, secondly, to an electrical and/or light and/or fluid power source.

11. The end tool as claimed in claim 8, wherein the surgical tool (11; 60) includes an interface layer (30; 66) adapted to be firmly attached below the functional layer (33; 70) and incorporating components (31, 32; 67, 68) for energy transfer between the external medium and the electronic layer.

12. The end tool as claimed in claim 8, wherein the surgical instrument (11; 60) includes pins (40) extending through superposed openings formed in the different layers of said tool and designed to be fixed into openings (7; 55) formed in the base layer (2; 51) of the tool-holder support (1; 50).

13. The end tool as claimed in claim 11, wherein it consists of a forceps formed by two tool-holder support/surgical instrument assemblies (1, 11, 1′, 11′), the tool-holder supports (1, 1′) of which are each provided on a prolongation of their base layer (2, 2′) with a lug (4, 4′) orthogonal to said base layer for the articulation of the forceps.

14. The end tool as claimed in claim 13, wherein
the functional layer (33) of each surgical tool incorporates at least one electrode (34, 35) flush with the upper face of said functional layer, the interface layer (30) including a conductive component (32) for supplying each electrode with energy.

15. The end tool as claimed in claim 11, wherein it consists of a bistoury having one blade or a scissors blade including a functional layer (70) in the form of a blade having a longitudinal side face with a beveled profile forming a longitudinal cutting edge.

16. The end tool as claimed in claim 15, wherein the functional layer (70) forms a bipolar blade provided with a thickness (72) made of electrically conductive material, the interface layer (66) including a conductive component (67) for supplying said conductive thickness with energy.

17. The end tool as claimed in claim 8, wherein it consists of a forceps formed by two tool-holder support/surgical instrument assemblies (1, 11, 1′, 11′), the tool-holder supports (1, 1′) of which are each provided on a prolongation of their base layer (2, 2′) with a lug (4, 4′) orthogonal to said base layer for the articulation of the forceps.

18. The end tool as claimed in claim 8, wherein it consists of a bistoury having one blade or a scissors blade including a functional layer (70) in the form of a blade having a longitudinal side face with a beveled profile forming a longitudinal cutting edge.

19. The end tool as claimed in claim 9, wherein the surgical tool (11; 60) includes a support layer (12; 61) designed to be fixed to the base layer (2; 51) of the tool-holder support (1; 50) and including a connector (14) for connection, firstly, to the connector of each electronic layer and, secondly, to an electrical and/or light and/or fluid power source.

20. The microsensor as claimed in claim 2, wherein:
clearance is provided between the fixed (104) and moving (106) blocks so as to allow relative translational displacement of these blocks (104, 106) in at least one direction, called the pressure direction, normal to the fixed (102) and moving (103) faces,
the restoring device (108) is elastically deformable under the effect of relative displacement of the blocks (104, 106) in each pressure direction,
it includes a measuring assembly (118, 119) adapted to measuring relative displacements of the blocks in each pressure direction and able to supply a signal representing these displacements and therefore representing the pressure stress applied between the moving face (103) and the fixed face (102).
